(19) United States
(12) Patent Application Publication
(10) Pub. No.: US 2007/0131136 A1
(43) Pub. Date: Jun. 14, 2007

(54) Composition And Process For Coloring Wood

(75) Inventors: Wenjin Zhang, Tonawanda, NY (US); Jun Zhang, Getzville, NY (US)

Correspondence Address: HODGSON RUSS LLP, ONE M & T PLAZA, SUITE 2000, BUFFALO, NY, 14203-2391, US

(73) Assignee: Osmose, Inc., Buffalo, NY (US)

(21) Appl. No.: 11/608,508

(22) Filed: Dec. 8, 2006

Related U.S. Application Data
(63) Continuation-in-part of application No. 11/299,522, filed on Dec. 12, 2005.
Application No. 11/299,522 is a continuation-in-part of application No. 11/126,839, filed on May 11, 2005.
Application No. 11/299,522 is a continuation-in-part of application No. 11/116,152, filed on Apr. 27, 2005.
(60) Provisional application No. 60/570,659, filed on May 13, 2004.
(60) Provisional application No. 60/565,585, filed on Apr. 27, 2004.

(51) Int. Cl. C09D 5/14; C09D 5/16; A61K 31/33; B05D 3/00; B32B 21/04; A61K 33/24; A01N 59/20; A61K 33/34; A01N 33/12; A01N 59/16; A61K 33/26; A61K 33/22

(52) U.S. Cl. 106/15.05; 106/18.3; 106/18.32; 106/18.33; 106/18.34; 106/18.35; 424/617; 424/630; 424/638; 424/642; 424/646; 424/650; 424/654; 424/657; 427/297; 428/537.1; 514/183; 514/642

(57) ABSTRACT

Provided is a method for coloring and, optionally, preserving wood and wood products. The method comprises application of pigment dispersions, and optionally biocide dispersions, to wood such that the wood is impregnated. A composition for coloring and, optionally, preserving wood is disclosed, comprising dispersions of micronized pigment and, optionally, micronized biocide.

This application is a continuation-in-part of U.S. Non-provisional application No. 11/299,522 filed on Dec. 12, 2005, which is a continuation-in-part of U.S. Non-provisional application No. 11/126,839 filed on May 11, 2005, which claims priority to U.S. Provisional application No. 60/570,659 filed on May 12, 2004, the disclosure of which is incorporated herein by reference; and is also a continuation-in-part of U.S. Non-provisional application No. 11/116,152 filed on Apr. 27, 2005, which claims priority to U.S. Provisional application No. 60/565,585 filed on Apr. 27, 2004, the disclosure of which is hereby incorporated by reference.

### FIELD OF THE INVENTION

This invention relates to a composition and method for both coloring and, optionally, preserving cellulosic products, such as wood, to improve their outdoor weathering properties and their resistance to rot- and decay-causing organisms or environmental agents. More particularly, the invention relates to a composition and method whereby coloring and preserving of wood may be accomplished in a single application step, or sequentially, in two separate application steps, which may be performed in either order.

### BACKGROUND OF THE INVENTION

Wood which is both colored and preserved is used extensively in the construction industry in applications including siding, fencing, and decking. It has long been desirable to produce wood products that have an aesthetically pleasing appearance and good outdoor weathering properties, and yet have resistance to attack by wood destroying agencies such as fungi, bacteria and insects.

Untreated wood, when exposed to an outdoor environment, is subject to bio-deterioration due to attack by decay fungi and insects. In addition, untreated wood is subject to photo-degradation which will cause yellowing, fading, graying and, over time, a darkening of the wood surface.

Traditionally, wood preservative solutions used by wood preservation industry to impart resistance to fungal and insect attack contain metals or metal complexes. Examples are chromated copper arsenate (CCA), alkaline copper quaternary ammonium compounds (ACQ) and others, such as those described in American Wood Preservers' Association Standards-2005. These preservative systems not only provide decay and termite resistance, but also provide protection against photo-degradation due to the presence of metal or metal complexes which can act as absorbers and/or blockers of ultraviolet radiation. Unfortunately, many of the metal-based preservatives impart an undesirable color to the wood.

Thus, the wood preservation industry is increasingly interested in non-traditional preservatives, such as organic preservatives or non-metal based preservatives. However, such preservatives generally weather poorly upon exposure to sunlight. In fact, wood which has been treated with these preservatives can weather as poorly as wood which has not undergone treatment.

Thus colorants have been used in conjunction with preservatives in an attempt to improve weathering properties of preserved wood.

One technique currently used to color wood is to paint the surface of the wood with oil or water based pigment paint coating. However, paint often will not adhere to preservative-treated wood, resulting in blistering or flaking of the coating in a short period of time.

Additionally, a critical failure of this and other coating methods is that they provide surface coloration which may wear away, or lose color due to dent or scratches or other physical damage to the surface, requiring additional treatment or servicing if long term weathering is desired.

Another technique currently used to color wood is the addition of water soluble dyes to the preservative solution thereby imparting color to treated wood products. However, water soluble dyes, such as acid dyes or cationic dyes, generally have poor lightfastness, generally fading or decomposing upon exposure to sunlight, particularly ultra violet (UV) wavelengths.

In view of the many shortcomings of the current methods of coloring and preserving wood, it is desirable to have a coloring and preserving system that provides an aesthetically pleasing appearance, long-term weathering performance, and resistance to biodeterioration. It is also desirable to have a coloring and preserving process which can, if desired, be completed in a single application step.

### SUMMARY OF THE INVENTION

Provided are wood colorant compositions that can be used with inorganic and organic wood preservatives. The colorant compositions comprise dispersions of inorganic and/or organic pigments in the form of micronized particles. The composition optionally additionally comprises inorganic and/or organic biocides, which may be present as dispersions, emulsions or in solution.

The compositions of the present invention can be used to color wood or in additional embodiments, to simultaneously color and preserve wood. In the art, “wood preservatives” and “biocides” are sometimes identified with inorganic (metal or metal salt) or organic compounds, respectively. However, herein, the terms “preservative” and “biocide” are used interchangeably to refer to both organic and inorganic compounds.

In one embodiment, the composition comprises a micronized inorganic pigment. In another embodiment, the composition comprises a micronized organic pigment. In yet another embodiment, the composition comprises one or more micronized inorganic and/or organic pigments and a micronized biocide. In further embodiments, the composition comprises one or more micronized inorganic and/or organic pigments and one or more inorganic and/or organic biocides. Dyes including acid dyes, basic dyes and direct dyes can optionally be added to the composition to further enhance the aesthetic appearance of the wood.

Also provided is a method for coloring and preserving wood simultaneously.

Also provided is a method for coloring and preserving wood comprising the step of impregnating wood with the compositions of the above embodiments.

Also provided is a method for coloring and preserving wood comprising the steps of:
1) impregnating the wood with a composition comprising a dispersion comprising one or more micronized pigments with or without addition of dyes; and
2) impregnating the wood with a composition comprising an inorganic or organic preservative biocide, optionally the biocide is micronized;
wherein the steps are conducted in either order.

Also provided is a method for imparting lightfast, uniform color to wood.

When wood is treated with the composition of the present invention, the pigment, and the preservative, if present, are carried beneath the surface of the wood, imparting long lasting color to the wood and preserving it from biological degradation.

Also provided is a method of impregnating color beneath the surface of wood to provide for long term application.

Also provided is a method for imparting color to wood which improves the outdoor weathering properties of wood.

Pigment formulations have been used to coat and paint wood. However, the present invention pertains to the coloring, and, optionally preserving of wood by impregnation with pigment, and optionally biocides, giving a preserved product having lightfast, non-flaking color. Impregnation into wood imparts to the wood excellent UV resistance, and thus, excellent weathering characteristics. Impregnation of colorants into wood also simplifies the coloring process and improves the efficiency and throughput of coloring and preserving wood compared to traditional painting and/or staining processes.

The pigment dispersion comprises micronized inorganic pigments, such as, for example, iron oxides including red, yellow, black and brown iron oxides, carbon black, cupric oxide, cuprous oxide, zinc oxides, titanium oxides and chrome oxides; and/or micronized organic pigments.

Also provided is a method for the treatment of wood or wood product with the compositions of the present invention

### BRIEF DESCRIPTION OF THE FIGURES

FIG. 1 depicts the anatomy of coniferous wood. A: Resin canal; B: Earlywood tracheids; C: Latewood tracheids; D: Bordered pits.

FIG. 2 depicts the border pit structure for coniferous woods.
RIGHT: Microscopic view of the cross section of a bordered pit.
LEFT: Torus in top view. The torus is supported by a net of radial fibril membrane, also called the margo. The flow of fluids between two tracheids through such a membrane is restricted by the size of the membrane openings. A: Pit aperture; B: Torus; C: Margo (microfibrils); D: Pit border.

FIG. 3 depicts the superior outdoor weathering of wood treated with tebuconazole and micronized red brown pigment formulation (3B) versus treatment with tebuconazole alone (3A).

FIG. 4 depicts the superior outdoor weathering of wood treated with quaternary ammonium compound and micronized green pigment formulation (4B) versus treatment with quaternary ammonium compound alone (4A).

FIG. 5 demonstrates the effect of QUV test on the wood samples treated with a preservative alone (dimethyl didecyl ammonium quat). Delignification and graying were observed after one month of QUV weathering.

FIG. 6 demonstrates the effect of QUV test on the wood samples treated with a preservative (azole based preservative) plus a light-brown iron oxide-based pigment formulation. Only slight color change observed after one month of QUV weathering.

### DETAILED DESCRIPTION OF THE INVENTION

The present invention provides compositions and methods for coloring, and optionally, preserving wood and wood products. The present invention also provides compositions and methods for coloring and preserving wood products simultaneously. The composition comprises a pigment dispersion, and optionally an organic or inorganic biocide/wood preservative. In some embodiments of the present invention, the pigments are red iron oxide, yellow iron oxide, black iron oxide, brown iron oxide, zinc oxide, titanium oxide, cuprous and cupric oxide, or carbon black. In additional embodiments, the composition additionally comprises metal compounds or metal complexes as a preservative, preferably copper compounds or copper complexes including copper carbonate, copper hydroxide, oxine copper, cuprous oxide and cupric oxide, optionally in a micronized form. It should be noted that pigment compounds may also have biocidal ability as well. In another embodiment, the preservative composition additionally comprises one or more organic or metal free biocides, preferably quaternary ammonium compounds, particularly didecyldimethylammonium chloride, didecyldimethylammonium carbonate/bicarbonate, alkyldimethylbenzylammonium chloride, alkyldimethylbenzylammonium carbonate/bicarbonate, didodecyldimethylammonium chloride, didodecyldimethylammonium carbonate/bicarbonate, didodecyldimethylammonium propionate, N,N-didecyl-N-methyl-poly(oxyethyl)ammonium propionate; imidazoles or triazoles, for example, tebuconazole, cyproconazole, propiconazole, hexaconazole, 1-[[2-(2,4-dichlorophenyl)-1,3-dioxolan-2-yl]methyl]-1H-1,2,4-triazole; cis-trans-3-chloro-4-[4-methyl-(2-(1H-1,2,4-triazol-1-ylmethyl)-1,3-dioxolan-2-yl]phenyl 4-chlorophenyl ether; (RS)-2-(4-fluorophenyl)-1-(1H-1,2,4-triazol-1-yl)-3-(trimethylsilyl)propan-2-ol; 2-(2,4-difluorophenyl)-(1H-1,2,4-triazole-1-yl)-3-trimethylsilyl-2-propanol; pyrethroids, such as, for example, bifenthrin, permethrin, cypermethrin; isothiazolone compounds, particularly 4,5-Dichloro-2-n-octyl-3(2H)-isothiazolone (RH-287), methylisothiazolinone, chloromethylisothiazolinone, 1,2-benzisothiazolin-3- one, 2-octyl-3-isothiazolone; Amine oxides, particularly Barlox 10, Barlox 12, Barlox 14 and Barlox 16; imidachloprid; chlorpyrifos; cyfluthrin; fipronil, chlorothalonil or combinations of the foregoing.

The present invention pertains to the use of pigment dispersions to color wood. The present invention also pertains to the use of pigment dispersions, in combination with organic and/or inorganic biocides, to color and preserve wood. The difference between pigments and dyes is generally understood by one of skill in the art. Pigments are generally more lightfast and have a greater resistance to UV degradation than dyes. Another difference is that pigments generally have little or no solubility in the medium in which they are applied. Thus, if the composition of the present invention is applied as an aqueous dispersion, the pigment in the composition is generally one which has little or no solubility in water. The present invention is primarily directed toward applications which include the use of pigments in an aqueous carrier. However, pigment dispersions in other carriers, such as polar or nonpolar organic carriers, including oil carriers, are within the ambit of the invention. Non-limiting examples of non-aqueous carriers which can be used are oil carriers such as, for example, mineral oil, linseed oil, soybean oil, AWPA p-9 oil, and other known in the art. In general, the term “pigment” as used herein refers to a wood coloring compound which, when applied in a carrier, has a solubility of less than 5g per 100 grams carrier in the chosen carrier, and preferably less than 1.0 g, 0.5 g, 0.1 g or 0.01 g per 100 grams of carrier (at 25° C.). Furthermore, the coloring compound also should have a water solubility of less than 5 g per 100 grams of water at 25° C., and preferably less than 1.0, 0.5, 0.1 or 0.01 g per 100 g of water.

The pigments which can be used in the compositions of the present invention include inorganic and organic pigments. Inorganic pigments include metal compounds of metals such as iron, zinc, titanium, lead, chromium, copper, cadmium, calcium, zirconium, cobalt, magnesium, aluminum, nickel, and other transition metals. Carbon black is also an inorganic pigment, which can be used in the present invention.

Some non-limiting examples of suitable inorganic pigments include: iron oxides, including red iron oxides, yellow iron oxides, black iron oxides and brown iron oxides; carbon black, iron hydroxide, graphite, black micaceous iron oxide aluminum flake pigments, pearlescent pigments, calcium carbonate, calcium phosphate, calcium oxide, calcium hydroxide, bismuth oxide, bismuth hydroxide, bismuth carbonate, copper carbonate, copper hydroxide, basic copper carbonate, cupric oxide, cuprous oxide, silicon oxide, zinc carbonate, barium carbonate, barium hydroxide, strontium carbonate, zinc oxide, zinc phosphate, zinc chromate, barium chromate, chrome oxide, titanium dioxide, zinc sulfide and antimony oxide, lead chrome, and cadmium pigments.

Preferred inorganic pigments are carbon black; graphite; iron oxides, including yellow, red, black and brown iron oxides; zinc oxide; titanium oxide and aluminum-based pigments, such as, for example Al₂O₃ and Al(OH)₃.

Non-limiting examples of organic pigments include Monoazo (arylide) pigments such as PY3, PY65, PY73, PY74, PY97 and PY98; Disazo (diarylide); Disazo condensation; Benzimidazolone; Beta Naphthol; Naphthol; metal-organic complexes; Isoindoline and Isoindolinone; Quinacridone; perylene; perinone; anthraquinone; diketo-pyrrolo pyrrole; dioxazine; triacrylcarbonium; the phthalocyanine pigments, such as cobalt phthalocyanine, copper phthalocyanine, copper semichloro- or monochlorophthalocyanine, copper phthalocyanine, metal-free phthalocyanine, copper polychlorophthalocyanine, phthalocyanine blue, etc.; organic azo compounds; organic nitro compounds; polycyclic compounds, such as phthalocyanine pigments, quinacridone pigments, perylene and perinone pigments; diketopyrrolo-pyrrole(DPP) pigments; thioindigo pigments; dioxazine pigments; quinophthalone pigments; triacrylcarbonium pigments, and Diaryl pyrrolopyroles, such as PR254.

Non-limiting examples of organic pigments which can be used in the present invention, grouped according to the color they produce (e.g. blues, blacks, greens, yellow, reds and browns), based on their color index include: Pigment Yellows (PY) 1, 11, 3, 12, 13, 14, 17, 81, 83, 65, 73, 74, 75, 97, 111, 120, 151, 154, 175, 181, 194, 93, 94, 95, 128, 166, 129, 153, 109, 110, 173, 139, 185, 138, 108, 24; Pigment Oranges (PO) 5, 36, 60, 62, 65, 68, 61, 38, 69, 31, 13, 34, 43, 51, 71, 73; Pigment Reds (PR) 3, 4, 171, 175, 176, 185, 208, 2, 5, 12, 23, 112, 146, 170, 48, 57, 60, 68, 144, 166, 214, 220, 221, 242, 122, 192, 202, 207, 209, 123, 149, 178, 179, 190, 224, 177, 168, 216, 226, 254, 255, 264, 270, 272; Pigment Violets (PV) 32, 19, 29, 23, 37; Pigment Browns 25, 23; Pigment Blacks 1, 31, 32, 20; Pigment Blues (PB)15, 15:1, 15:2, 15:3, 15:4, 15:6, 16, 60; and Pigment Greens (PG) 7, 36.

It is preferred that the dispersion particle size distributions (or emulsion droplet size distribution, if applicable) contain particles (or droplets) of micronized size. The term “micronized” as used herein means a particle size in the range of 0.001 to 25 microns.

It should be understood that “micronized” does not refer only to particles which have been produced by the finely dividing, such as by mechanical grinding through media mill, of materials which are in bulk or other form. Micronized particles can also be formed by other mechanical, chemical or physical methods, such as, for example, pulverization process, formation in solution or in situ, with or without a seeding agent, grinding or impinging jet. The term “particle size” refers to the largest axis of the particle. In the case of a generally spherical particle, the largest axis is the diameter.

The formulations of micronized inorganic and/or organic pigments can be obtained by grinding the pigments, optionally wetted or present as a dispersion, to the desired particle size using a grinding mill. Other fine particle dividing methods known in the art can also be used, such as high speed, high shear mixing or agitation. The resulting particulate additive can be mixed with an aqueous liquid carrier to form a solution of dispersed additive particles.

Optionally, the solution can comprise a thickener, such as, for example, a cellulose derivative, as is known in the art and/or resin binder, such as polyacrylic, polyurethane, and other known in the art.

The particles are preferably dispersed and stabilized in the presence of dispersant(s). Dispersants function in micronized particle systems by 1) replacing the air around the particle surface and thus wetting the particle; 2) breaking down and/or preventing formation of particle agglomerates; and 3) stabilizing ground/reduced particles and preventing flocculation during storage. Dispersants can be divided into classes, two of which are polymeric dispersants and conventional low molecular surfactant type of dispersants. Polymeric dispersants generally possess a structure with pigment-affinic groups and a polymer chain and stabilizes the pigment particles through steric hindrance, while surfactant type dispersants generally possesses a hydrophilic group and a hydrophobic group and it stabilizes pigment particles through electrostatic mechanism.

Pigment stabilization can be particularly important when using micronized particles in wood treating processes. It not only requires a stabilized particle during storage, but also requires that particles maintain stability during repetitive treatments in a commercial treating plant. During repetitive treating processes, pH change, wood extractives, wood sugars and contaminants from wood surface and dirt from air can all affect the function of dispersants and hence the dispersion particle stability. Many surfactant-type low molecular weight dispersants can provide particles with short-term stability; in addition, surfactant-type low molecular weight dispersants are more sensitive to treatment environmental variables, such as the wood extractives, wood sugars, water quality, and degree of shear experienced during treatment. As a result, surfactant type dispersants are generally not preferred for the current application in preparing pigment dispersions. The particles can become unstable during long-term storage and during treating process and result in agglomeration and aggregation into large particles. Such agglomerates can impede treatment and leave a sludge on the surface of treated wood.

We have surprisingly found that polymeric dispersants can not only provide long-term stability of pigment dispersion particles, but also impart a high degree of stability during repetitive treatment processes. Generally, the weight average molecular weight of the polymeric dispersants varies from a few thousand to 100,000 or even more.

Non-limiting examples of polymeric dispersant classes which can be used in the compositions of the present invention include acrylic copolymers, aqueous solution of copolymers with pigment affinity groups, polycarboxylate ether, modified polyacrylate or modified polyacrylate with groups of high pigment affinity, acrylic polymer emulsions, modified acrylic polymers, poly carboxylic acid polymers and their salts, modified poly carboxylic acid polymers and their salts, fatty acid modified polyester, aliphatic polyether or modified aliphatic polyether, solution of polycarboxylate ether, phosphate esters, phosphate ester modified polymers, polyglycol ethers or modified polyglycol ethers, polyetherphosphate, modified maleic anhydride/styrene copolymer, sodium polyacrylate, sodium polymethacrylate, lignin, modified lignin and the like; modified polyether or polyester with pigment affinic groups; fatty acid derivatives; urethane copolymer or modified urethane copolymer, polyetherphosphate, modified maleic anhydride/styrene copolymer, modified polycarboxylic acid or its derivatives, acrylic acid/maleic acid copolymer, polyvinyl pyrrolidone or modified polyvinyl pyrrolidone, sulfonates or sulfonate derivatives, polymeric alkoxylate or its derivatives, or modified lignin and the like. If desired, a stabilizer as is known in the art can be used. Other dispersants can be found in 2004 McCutcheon's Functional Materials (North American Edition).

We have found that polymeric dispersants, especially modified polycarboxylate ether, modified poly carboxylic acid polymers and their salts, solutions of polycarboxylate ethers; modified polyether or polyester with pigment affinic groups, perform well with inorganic pigment compounds, particularly iron oxides, in providing wetting, dispersing, storage stabilization and stability during treatment process. We have also found that polymeric dispersants, particularly modified maleic anhydride/styrene copolymer or acrylic acid/maleic acid copolymer, perform well with organic pigments in providing wetting, dispersing, storage stabilization and stability during repetitive treating process.

For inorganic pigments, such as iron oxides, the level of dispersant is preferably in the range of from about 0.1 to 180% of the weight of the pigment compounds, and in other embodiments, in the range of 1 to 80%, 5 to 60%, and 10 to 30%. For organic pigments, the level of dispersant is preferably in the range of from about 1 to 200% of the weight of the pigment compounds, and in other embodiments in the range of 5 to 100%, 10 to 80%, and 30 to 70%.

If desired, a wetting agent can be used in the preparation of the compositions of the present invention. The level of wetting agent is preferably in the range of from about 0.1 to 50% of the weight of the pigment, and in other embodiments in the range of 0.5 to 10%, and 0.5 to 5%.

The composition of the present invention can be a concentrate or a preparation which is ready to apply to wood. In general, the total pigment concentrate is in the range of from 1 wt % to 80 wt %, based on weight of the composition, and preferably in the range of from 5 to 70 wt %, and more preferably in the range of from 30 to 65 wt %.

In the composition of the present invention, it is preferable that the pigment dispersion be present in the treating liquid applied to wood in amounts in the range of from 0.005 to 50 wt % of the solution, with a preferred range of 0.01 to 20%, and a more preferred range of 0.05 to 10%, and an even more preferred range of 0.1 to 1.0%.

A preferred method of preparing the pigment particles is by grinding. An exemplary method involves the formation of a slurry comprising a dispersant, a carrier, and a powdered pigment having a particle size in the range of from 1 micron to 500 microns, and optionally, a defoamer. The slurry is transferred to a grinding mill which is prefilled with a grinding media having a size in the range of from 0.05 mm to 5 mm, and preferably between 0.1 and 1 mm. The media can be one or more of many commercially available types, including but not limited to steel shots, carbon steel shots, stannous steel shots, chrome steel shots, ceramic (for example, alumina-containing); zirconium-based, such as zirconia, zirconium silicate, zirconium oxide stabilized zirconia such as yttrium-stabilized zirconia and ceria-stabilized zirconia; stabilized magnesium oxide; stabilized aluminum oxide, etc. The medium preferably occupies 50% to 99% of the grinding chamber volume, with 75 to 95% preferred, and 80 to 90% more preferred. The bulk density of the grinding media is preferably in the range of from 0.5 kg/l to 10 kg/l, and more preferably in the range of from 2 to 5 kg/l. Agitation speed, which can vary with the size of the grinder, is generally in the range of from 1 to 5000 rpm, but can be higher or lower. Lab and commercial grinders generally run at different speeds. A set up which involves a transfer pump which repeatedly cycles the slurry between the mill and a storage tank during grinding is convenient. The transfer pump speed varies from 1 to 500 rpm, and the speeds for lab and commercial grinders can be different. During grinding, defoamer can be added if foaming is observed. During grinding, particle size distribution can be analyzed, and once particle size is within the desired specification, grinding is stopped.

In the compositions of the present invention, in some embodiments, it preferred that at least 98% of the particles (by weight) have a diameter less than 10 microns, less than 5 microns or less than 1 micron.

The penetration of the pigment dispersion formulation into the cellular structure of wood or other cellulose-based material is dependent upon particle size considerations. If the inorganic/organic pigments used in formulating the dispersion formulation disclosed herein have a particle size in excess of 25 microns, the particles may be filtered by the surface of the wood and thus may not be uniformly distributed within the cell and cell wall.

In addition to the pigment dispersions, the present invention may comprise an inorganic and/or organic biocide component. This component may be micronized, emulsified, or present in solution. The above particle size considerations apply to the total particulate content, whether the particles are pigments or other particulate composition components. Although it is desirable to prepare separate concentrates of micronized pigments and micronized biocides and combine them to make treating compositions in the treating plant, a blended concentrate of micronized pigments and, optionally, micronized biocides and treating compositions can be obtained by direct dilution.

Non-limiting examples of inorganic biocides include materials such as metal complexes and metal compounds, as well as alkaline metal complexes and other metal complexes. Suitable metals include copper, arsenic, zinc, silver, cadmium, nickel, bismuth, lead and chromium, with copper being preferred. Suitable metal compounds and complexes can be obtained as concentrates, such as copper oxides, copper carbonate, copper omadine, copper 8-hydroxyquinolate (oxine copper).

The metal compounds, such as copper compounds, can also be in micronized particulate form when used with pigment dispersion. The preparation of the micronized metal compounds and the particle size range and distribution are similar to those of micronized pigments.

In addition to or instead of metal compounds and/or metal complexes, the present invention can also comprise organic biocidal compounds.

Some non-limiting examples of organic biocides are listed as follows:

Aliphatic Nitrogen Fungicides

butylamine; cymoxanil; dodicin; dodine; guazatine; iminoctadine
   Amide Fungicides
carpropamid; chloraniformethan; cyazofamid; cyflufenamid; diclocymet; ethaboxam; fenoxanil; flumetover; furametpyr; prochloraz; quinazamid; silthiofam; triforine benalaxyl; benalaxyl-M; furalaxyl; metalaxyl; metalaxyl-M; pefurazoate; benzohydroxamic acid; tioxymid; trichlamide; zarilamid; zoxamide cyclafuramid; furmecyclox dichlofluanid; tolylfluanid benthiavalicarb; iprovalicarb benalaxyl; benalaxyl-M;boscalid; carboxin; fenhexamid; metalaxyl; metalaxyl-M metsulfovax; ofurace; oxadixyl; oxycarboxin;pyracarbolid; thifluzamide; tiadinil benodanil; flutolanil; mebenil; mepronil; salicylanilide; tecloftalam fenfuram; furalaxyl; furcarbanil; methfuroxam flusulfamide
   Antibiotic Fungicides
aureofungin; blasticidin-S; cycloheximide; griseofulvin; kasugamycin;natamycin; polyoxins; polyoxorim; streptomycin; validamycin azoxystrobin dimoxystrobin fluoxastrobin kresoxim-methyl metominostrobin orysastrobin picoxystrobin pyraclostrobin trifloxystrobin
   Aromatic Fungicides
biphenyl chlorodinitronaphthalene chloroneb chlorothalonil cresol dicloran hexachlorobenzene pentachlorophenol quintozene sodium pentachlorophenoxide tecnazene
   Benzimidazole Fungicides
benomyl carbendazim chlorfenazole cypendazole debacarb fuberidazole mecarbinzid rabenzazole thiabendazole
   Benzimidazole Precursor Fungicides
furophanate thiophanate thiophanate-methyl
   Benzothiazole Fungicides
bentaluron chlobenthiazone TCMTB
   Bridged Diphenyl Fungicides
bithionol dichlorophen diphenylamine
   Carbamate Fungicides
benthiavalicarb furophanate iprovalicarb propamocarb thiophanate thiophanate-methyl benomyl carbendazim cypendazole debacarb mecarbinzid diethofencarb
   Conazole Fungicides
climbazole clotrimazole imazalil oxpoconazole prochloraz triflumizole azaconazole bromuconazole cyproconazole diclobutrazol difenoconazole diniconazole diniconazole-M epoxiconazole etaconazole fenbuconazole fluquinconazole flusilazole flutriafol furconazole furconazole-cis hexaconazole imibenconazole ipconazole metconazole myclobutanil penconazole propiconazole prothioconazole quinconazole simeconazole tebuconazole tetraconazole triadimefon triadimenol triticonazole uniconazole uniconazole-P
   Dicarboximide Fungicides
famoxadone fluoroimide chlozolinate dichlozoline iprodione isovaledione myclozolin procymidone vinclozolin captafol captan ditalimfos folpet thiochlorfenphim
   Dinitrophenol Fungicides
binapacryl dinobuton dinocap dinocap-4 dinocap-6 dinocton dinopenton dinosulfon dinoterbon DNOC
   Dithiocarbamate Fungicides
azithiram carbamorph cufraneb cuprobam disulfiram ferbam metam nabam tecoram thiram ziram dazomet etem milneb mancopper mancozeb maneb metiram polycarbamate propineb zineb
   Imidazole Fungicides
cyazofamid fenamidone fenapanil glyodin iprodione isovaledione pefurazoate triazoxide
   Morpholine Fungicides
aldimorph benzamorf carbamorph dimethomorph dodemorph fenpropimorph flumorph tridemorph
   Organophosphorus Fungicides
ampropylfos ditalimfos edifenphos fosetyl hexylthiofos iprobenfos phosdiphen pyrazophos tolclofos-methyl triamiphos
   Oxathiin Fungicides
carboxin oxycarboxin
   Oxazole Fungicides
chlozolinate dichlozoline drazoxolon famoxadone hymexazol metazoxolon myclozolin oxadixyl vinclozolin
   Pyridine Fungicides
boscalid buthiobate dipyrithione fluazinam pyridinitril pyrifenox pyroxychlor pyroxyfur
   Pyrimidine Fungicides
bupirimate cyprodinil diflumetorim dimethirimol ethirimol fenarimol ferimzone mepanipyrim nuarimol pyrimethanil triarimol
   Pyrrole Fungicides
fenpiclonil fludioxonil fluoroimide
   Quinoline Fungicides
ethoxyquin halacrinate 8-hydroxyquinoline sulfate quinacetol quinoxyfen
   Quinone Fungicides
benquinox chloranil dichlone dithianon
   Quinoxaline Fungicides
chinomethionat chlorquinox thioquinox
   Thiazole Fungicides
ethaboxam etridiazole metsulfovax octhilinone thiabendazole thiadifluor thifluzamide
   Thiocarbamate Fungicides
methasulfocarb prothiocarb
   Thiophene Fungicides
ethaboxam silthiofam
   Triazine Fungicides
anilazine
   Triazole Fungicides
bitertanol fluotrimazole triazbutil
   Urea Fungicides
bentaluron pencycuron quinazamid
   Other Fungicides
acibenzolar acypetacs allyl alcohol benzalkonium chloride benzamacril bethoxazin carvone chloropicrin DBCP dehydroacetic acid diclomezine diethyl pyrocarbonate fenaminosulf fenitropan fenpropidin formaldehyde furfural hexachlorobutadiene iodomethane isoprothiolane methyl bromide methyl isothiocyanate metrafenone nitrostyrene nitrothal-isopropyl OCH 2 phenylphenol phthalide piperalin probenazole proquinazid pyroquilon sodium orthophenylphenoxide spiroxamine sultropen thicyofen tricyclazole, methyl isothiocyanate

Preferred insecticides which can be mixed micronized metal formulations are:

Antibiotic Insecticides

allosamidin thuringiensin spinosad abamectin doramectin emamectin eprinomectin ivermectin selamectin milbemectin milbemycin oxime moxidectin
   Botanical Insecticides
anabasine azadirachtin d-limonene nicotine pyrethrins cinerins cinerin I cinerin II jasmolin I jasmolin II pyrethrin I pyrethrin II quassia rotenone ryania sabadilla
   Carbamate Insecticides
bendiocarb carbaryl benfuracarb carbofuran carbosulfan decarbofuran furathiocarb dimetan dimetilan hyquincarb pirimicarb alanycarb aldicarb aldoxycarb butocarboxim butoxycarboxim methomyl nitrilacarb oxamyl tazimcarb thiocarboxime thiodicarb thiofanox allyxycarb aminocarb bufencarb butacarb carbanolate cloethocarb dicresyl dioxacarb EMPC ethiofencarb fenethacarb fenobucarb isoprocarb methiocarb metolcarb mexacarbate promacyl promecarb propoxur trimethacarb XMC xylylcarb
   Dinitrophenol Insecticides
dinex dinoprop dinosam DNOC cryolite sodium hexafluorosilicate sulfluramid
   Formamidine Insecticides
amitraz chlordimeform formetanate formparanate
   Fumigant Insecticides
acrylonitrile carbon disulfide carbon tetrachloride chloroform chloropicrin para-dichlorobenzene 1,2-dichloropropane ethyl formate ethylene dibromide ethylene dichloride ethylene oxide hydrogen cyanide iodomethane methyl bromide methylchloroform methylene chloride naphthalene phosphine sulfuryl fluoride tetrachloroethane
   Insect Growth Regulators
bistrifluron buprofezin chlorfluazuron cyromazine diflubenzuron flucycloxuron flufenoxuron hexaflumuron lufenuron novaluron noviflumuron penfluron teflubenzuron triflumuron epofenonane fenoxycarb hydroprene kinoprene methoprene pyriproxyfen triprene juvenile hormone I juvenile hormone II juvenile hormone III chromafenozide halofenozide methoxyfenozide tebufenozide α-ecdysone ecdysterone diofenolan precocene I precocene II precocene III dicyclanil
   Nereistoxin Analogue Insecticides
bensultap cartap thiocyclam thiosultap flonicamid clothianidin dinotefuran imidacloprid thiamethoxam nitenpyram nithiazine acetamiprid imidacloprid nitenpyram thiacloprid
   Organochlorine Insecticides
bromo-DDT camphechlor DDT pp′-DDT ethyl-DDD HCH gamma-HCH lindane methoxychlor pentachlorophenol TDE aldrin bromocyclen chlorbicyclen chlordane chlordecone dieldrin dilor endosulfan endrin HEOD heptachlor HHDN isobenzan isodrin kelevan mirex
   Organophosphorus Insecticides
bromfenvinfos chlorfenvinphos crotoxyphos dichlorvos dicrotophos dimethylvinphos fospirate heptenophos methocrotophos mevinphos monocrotophos naled naftalofos phosphamidon propaphos schradan TEPP tetrachlorvinphos dioxabenzofos fosmethilan phenthoate acethion amiton cadusafos chlorethoxyfos chlormephos demephion demephion-O demephion-S demeton demeton-O demeton-S demeton-methyl demeton-O-methyl demeton-S-methyl demeton-S-methylsulphon disulfoton ethion ethoprophos IPSP isothioate malathion methacrifos oxydemeton-methyl oxydeprofos oxydisulfoton phorate sulfotep terbufos thiometon amidithion cyanthoate dimethoate ethoate-methyl formothion mecarbam omethoate prothoate sophamide vamidothion chlorphoxim phoxim phoxim-methyl azamethiphos coumaphos coumithoate dioxathion endothion menazon morphothion phosalone pyraclofos pyridaphenthion quinothion dithicrofos thicrofos azinphos-ethyl azinphos-methyl dialifos phosmet isoxathion zolaprofos chlorprazophos pyrazophos chlorpyrifos chlorpyrifos-methyl butathiofos diazinon etrimfos lirimfos pirimiphos-ethyl pirimiphos-methyl primidophos pyrimitate tebupirimfos quinalphos quinalphos-methyl athidathion lythidathion methidathion prothidathion isazofos triazophos azothoate bromophos bromophos-ethyl carbophenothion chlorthiophos cyanophos cythioate dicapthon dichlofenthion etaphos famphur fenchlorphos fenitrothion fensulfothion fenthion fenthion-ethyl heterophos jodfenphos mesulfenfos parathion parathion-methyl phenkapton phosnichlor profenofos prothiofos sulprofos temephos trichlormetaphos-3 trifenofos butonate trichlorfon mecarphon fonofos trichloronat cyanofenphos EPN leptophos crufomate fenamiphos fosthietan mephosfolan phosfolan pirimetaphos acephate isocarbophos isofenphos methamidophos propetamphos dimefox mazidox mipafox
   Oxadiazine Insecticides
indoxacarb
   Phthalimide Insecticides
dialifos phosmet tetramethrin
   Pyrazole Insecticides
acetoprole ethiprole fipronil tebufenpyrad tolfenpyrad vaniliprole
   Pyrethroid Insecticides
acrinathrin allethrin bioallethrin barthrin bifenthrin bioethanomethrin cyclethrin cycloprothrin cyfluthrin beta-cyfluthrin cyhalothrin gamma-cyhalothrin lambda-cyhalothrin cypermethrin alpha-cypermethrin beta-cypermethrin theta-cypermethrin zeta-cypermethrin cyphenothrin deltamethrin dimefluthrin dimethrin empenthrin fenfluthrin fenpirithrin fenpropathrin fenvalerate esfenvalerate flucythrinate fluvalinate tau-fluvalinate furethrin imiprothrin metofluthrin permethrin biopermethrin transpermethrin phenothrin prallethrin profluthrin pyresmethrin resmethrin bioresmethrin cismethrin tefluthrin terallethrin tetramethrin tralomethrin transfluthrin etofenprox flufenprox halfenprox protrifenbute silafluofen
   Pyrimidinamine Insecticides
flufenerim pyrimidifen
   Pyrrole Insecticides
chlorfenapyr
   Tetronic Acid Insecticides
spiromesifen
   Thiourea Insecticides
diafenthiuron
   Urea Insecticides
flucofuron sulcofuron
   Other Insecticides
closantel crotamiton EXD fenazaflor fenoxacrim hydramethylnon isoprothiolane malonoben metoxadiazone nifluridide pyridaben pyridalyl rafoxanide triarathene triazamate

Preferred bactericides include:
bronopol cresol dichlorophen dipyrithione dodicin fenaminosulf formaldehyde hydrargaphen 8-hydroxyquinoline sulfate kasugamycin nitrapyrin octhilinone oxolinic acid oxytetracycline probenazole streptomycin tecloftalam thiomersal

Preferred biocides include: azole compounds, such as tebuconazole; cyproconazole; propiconazole; hexaconazole, 1-[[2-(2,4-dichlorophenyl)-1,3-dioxolan-2-yl]methyl]-1H-1,2,4-triazole; cis-trans-3-chloro-4-[4-methyl-2-(1H-1,2,4-triazol-1-ylmethyl)-1,3-dioxolan-2-yl]phenyl 4-chlorophenyl ether; (RS)-2-(4-fluorophenyl)-1-(1H-1,2,4-triazol-1-yl)-3-(trimethylsilyl)propan-2-ol; 2-(2,4-difluorophenyl)-1-(1H-1,2,4-triazole-1-yl)-3-trimethylsilyl-2-propanol; isothiazolone compounds, such as methylisothiazolinone; chloromethylisothiazolinone; 4,5-Dichloro-2-n-octyl-3(2H)-isothiazolone; 1,2-benzisothiazolin-3-one; 2-octyl-3-isothiazolone; imidachloprid; fipronil; cyfluthrin; bifenthrin; permethrin; cypermethrin; and chlorpyrifos; iodopropynyl butylcarbamate (IPBC); chlorothalonil; 2-(thiocyanatomethylthio) benzothiazole; alkoxylated diamines and carbendazim.

Organic biocides also include quaternary ammonium compounds disclosed in the present invention have the following structures: 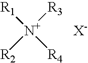
where R1, R2, R3, and R4 are independently selected from alkyl or aryl groups and X⁻ selected from chloride, bromide, iodide, carbonate, bicarbonate, borate, carboxylate, hydroxide, sulfate, acetate, laurate, or any other anionic group.

Preferred quaternary ammonium compounds include didecyldimethylammonium chloride; didecyldimethylammonium carbonate/bicarbonate; alkyldimethylbenzylammonium chloride; alkyldimethylbenzylammonium carbonate/bicarbonate; didodecyldimethylammonium chloride; didodecyldimethylammonium carbonate/bicarbonate; didodecyldimethylammonium propionate; N,N-didecyl-N-methyl-poly(oxyethyl)ammonium propionate.

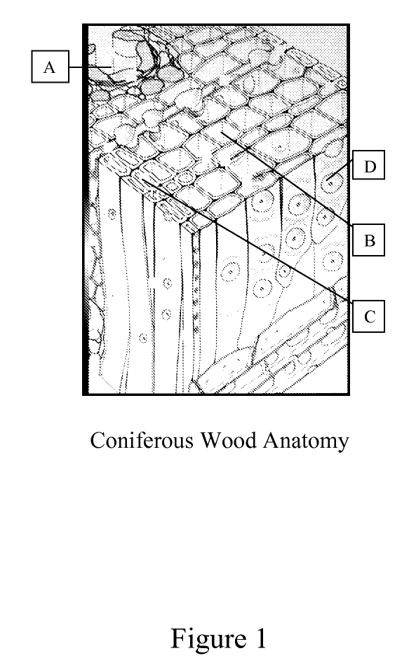

Without desiring to be bound by theory, penetration of the micronized dispersion formulation into wood takes place because particles migrate into or are taken up by tracheids in the wood. FIG. 1 shows the physiological structure of wood. As shown in FIG. 1, the primary entry and movement of fluids through wood tissue occurs primarily through the tracheids and border pits. Fluids are transferred between wood cells by means of border pits, which are generally smaller in diameter than the tracheids. When wood is treated with micronized pigment dispersion, if the particle size of the pigment is less than the diameter of the pit openings, a complete penetration and a uniform distribution of micronized preservative in wood is expected. Wood tracheids generally have diameters of around 30 microns, and good penetration can be achieved by the use of particles having long axis dimensions (“particle size” which are less than the tracheid diameters of the wood or wood product to be treated.

Studies by Mercury-Porosimetry technique indicated that the overall diameter of the border pit chambers typically varies from a several microns up to thirty microns while, the diameter of the pit openings (via the microfibrils) typically varies from several hundredths of a micron to several microns. FIG. 2 depicts the border pit structure for coniferous woods. Thus, in order to maximize penetration and uniformity of distribution of the particulate composition, the particle size should be such that it can travel through the pit openings.

The size of the particles used in the dispersion formulation disclosed herein can be micronized, i.e., with a long axis dimension between 0.001-25 microns. In another embodiment, the particle size is between 0.001-10 microns. In another embodiment, the particle size is between 0.01-5 microns. In yet another embodiment, the particle size is between 0.01 to 2 microns. If superior uniformity of penetration is desired, particle size of the additive used in the dispersion formulation disclosed herein should be between 0.05-1 microns.

It should be noted that the above does not exclude the presence of particles outside the stated ranges. However, particles which are too large can clog the wood, preventing it from taking in other particles. Thus particle size distributional parameters can affect the uniformity of particle distribution in the wood, as well as the leaching properties of treated wood. It is thus preferable to use particle size distributions which contain relatively few particle sizes outside the range of 0.001 to 25 microns. It is preferred that no more than 20 weight percent of the particles have diameters which are greater than 25 microns. Regardless of the foregoing recommendations, it is generally preferred that at least 60%, and more preferably, at least 80 wt % of the particles have a diameter in the range of 0.001 to 25 microns. In more preferred embodiments, greater than 85, 90, 95 or 99 wt percent particles are in the range of 0.001 to 25 microns. Depending on the degree of penetration desired, greater than 5, 10, 20 or 50 wt % of the particles can be less than 5, 1 or 0.5 microns.

For increased degree of penetration and uniformity of distribution, at least 50 wt % of the particles should have diameters which are less than 10 microns. More preferred are particle distributions which have at least 80, 90, 95, or 99 wt % of the particles with sizes of less than 10 microns. In an additional embodiment, at least 60 wt % of the particles should have diameters which are less than 1 micron. More preferred are particle distributions which have at least 80, 90, 95, or 99 wt % of the particles with sizes of less than 1 micron.

In order to further improve the weathering properties and the lightfastness of the pigment treated wood or further improve the adhesion of pigment particles to wood, a resin binder is often used in the composition. Examples of resin binders which can be used include polyurethane, polyester, polyvinyl alcohol, polyamide, epoxy, acrylic polymers, vinyl polymers (including polymers made from ethylenically unsaturated monomers such as polybutene), cellulosic derivatives, oligomers and natural polymers, can be either added to the pigment dispersion or added to the final treating composition. Examples of resin binders include:
1). Natural resins, such as fatty vegetable oils, mixtures of complex cyclic or aromatic acids, fish oils, and the like.
2). Vinyl based resins, such as polyethylene, polypropylene, polyvinyl chloride, polyvinyl alcohol, polystyrene, polyalpha methyl styrene, polyvinyl acetate, polymethyl methacrylate, polyacrylonitrile, polyvinyl ethyl ether, polyvinylidene fluoride and the like.
3). Acrylic resins, such as polyacrylic acid, polymethacrylic acid, polyethyl acrylate, polymethyl methacrylate, polylauryl methacrylate, poly2-hydroxyethyl acylate, polyglycidal methaacylate, polyacrylamide, polyhexane diol diacylate, polytrimethylol propane triacrylate, polycarboxylic acid, and the like.
4). Hydrocarbon resins and bituminous binders, such as petroleum oil-derived hydrocarbon resins, terpene resins, ketone resins, asphltite, petroleum asphalts, bituminous mastics, asphaltic hybrids, and the like.
5). Cellulosic resins, such as nitrocellulose, cellulose acetate, cellulose acetate butyrate, ethylcellulose, carboxylmethyl cellulose, methyl cellulose, ethyl cellulose, hydroxyethyl cellulose, hydroxypropyl cellulose, methyl hydroxyethyl cellulose, and the like.
6). Vegetable oils and modified vegetable oils, such as castor oil, linseed oil, tung oil, soya oil, tall oil, safflower oil, fish oil, and the like.
7). Alkyd resins, such as polyethylene glycol, polyneopentyl glycol, polyglycerol, polypentaerythritol, polybenzoic acid, polyabietic acid, polyterephthalic acid, polytrimellitic anhydride, polyisophthalic acid, polyamide-modified alkyds, and the like.
8). Polyester and polyesteramide resins, such as polyethylene terephthalate. The polyesters can be obtained, as well known, by polycondensation of dicarboxylic acids with polyols, in particular diols. The polyesteramides can be obtained in a similar manner to that for the polyesters, by polycondensation of diacids with diamines or amino alcohols, and the like.
9). Formaldehyde resins, such as phenolic resins including phenolic novolacs, phenolic resoles, phenolic epoxies, and phenolic modified rosins, amino resins including urea formaldehyde resins, melamine formaldehyde resins and hexamethoxymethyl melamine resins, and the like.
10). Epoxy resins, such as bisphenol A based epoxy resins, bisphenol F epoxy resins, polyglycol epoxy resins, cardanol-based epoxies and brominated epoxies, and the like.
11). Polyurethanes: The polyurethanes may be chosen from anionic, cationic, nonionic or amphoteric polyurethanes, acrylic polyurethanes, polyurethane-polyvinylpyrrolidones, polyester-polyurethanes, polyether-polyurethanes, polyureas, polyurea-polyurethanes and mixtures thereof. The polyurethane can be, for example, an aliphatic, cycloaliphatic or aromatic polyurethane, polyurea/urethane or polyurea copolymer containing, alone or as a mixture: one sequence of linear or branched aliphatic and/or cycloaliphatic and/or aromatic polyester origin, and/or one sequence of aliphatic and/or cycloaliphatic and/or aromatic polyether origin. The polyurethanes can also be obtained from branched or unbranched polyesters, or from alkyds containing labile hydrogens which are modified by reaction with a diisocyanate and a difunctional (for example dihydro, diamino or hydroxyamino) organic compound, in addition containing either a carboxylic acid or carboxylate group, or a sulphonic acid or sulphonate group, or alternatively a tertiary amine group or a quaternary ammonium group, and the like.
12). Silicone Resins: the silicone compounds, in emulsion, are preferably polyorganosiloxanes, which can be provided in the form of oils, in particular, volatile or nonvolatile silicone oil, of gums, of resins, of pasty products or of waxes, or their mixtures. The silicone gums, waxes and resins can be mixed with silicone oils in which they may be dissolved, the mixture being in the form of an oil-in-water emulsion.
13). Silicate resins, such as alkali silicate binders, alkyl silicate binders, cementitious binders and zinc rich silicate binders, and the like.

In the composition of the present invention, the ratio of the resin content to the pigment solid content in the treating composition ranges from about 0.001:1 to about 1000:1, and preferably ranges from about 0.01:1 to about 10:1, and more preferably range from 0.05:1 to about 1:1.

In the present invention dyes can optionally be added to the pigment compositions or preservative composition or treating compositions to obtain the desirable color and enhance the color richness of the treated wood. Dyes can be basic dyes (or cationic dyes), acid dyes, direct dyes, azoic dyes, sulfur dyes, vat dyes, and reactive dyes. Preferred dyes are basic dyes.

Non-limiting examples of basic dyes are: derivatives of diphenylmethane; triphenylmethane or acridine; thiazine, oxazine, or azine dyes; xanthene basic dyes, basic dyes containing azo groups, and basic dyes containing a pendant cation, a delocalized charge, or a heterocylic ring which contains a quaternary nitrogen atom; 2-(((4-Methoxyphenyl)methylhydrazono) methyl)-1,3,3-trimethyl-3H-indolium methyl sulphate; 2-(2-(4-((2-Chloroethyl) methylamino)phenyl)vinyl)-1,3,3-trimethyl-3H-indolium chloride; 3,7-Bis(dimethylamino)phenothiazin-5-ium chloride; 7-(Dimethylamino)-6-nitro-3H-phenothiazin-3-ylidene)dimethylammonium chloride; Methanaminium N-[4-[[4-(dimethylamino)phenyl]phenylmethylene]-2,5-cyclohexadien-1-ylidene]-N-methyl-, chloride; 4-((4-aminophenyl)(4-imino-2,5-cyclohexadien-1-ylidene)methyl)-2-methyl-benzenamine; 4,4′-((4-imino-2,5-cyclohexadien-1-ylidene)methylene)dianiline monohydrochloride; 1,3-Benzenediamine, 4,4′-(1,3-phenylenebis(azo))bis-, dihydrochloride; 3-Methyl-2-((1-methyl-2-phenyl-1H-indol-3-yl)azo)thiazolium chloride; (2-((4-((2-Chloro-4-nitrophenyl)azo)phenyl)ethylamino) ethyl)trimethylammonium methyl sulphate; 2-(((1,3-Dihydro-1,3-dimethyl-2H-benzimidazol-2-ylidene)methyl)azo)-3-methylbenzothiazolium methyl sulphate; (2-((4-((2-Chloro-4-nitrophenyl)azo)phenyl)ethylamino)ethyl)trimethylammonium; 4-((2-Chlorophenyl)(4-(ethylimino)-3-methylcyclohexa-2,5-dien-1-ylidene)methyl)-N-ethyl-o-toluidine monohydrochloride; 2-(2-(4-((2-Chloroethyl)ethylamino)-o-tolyl)vinyl)-1,3,3-trimethyl-3H-indolium chloride.

Non-limiting examples of acid dyes are: derivatives of triphenylmethane, derivatives of xanthene, nitrated aromatic compounds, acid dyes containing one or more azo groups, pyrazolone azo dyes, derivatives of anthraquinone dyes, derivatives of phthalocyanine dyes. Non-limiting examples of direct dyes are: sulphonated azo compounds and metal complex direct dyes.

In the composition of the present invention, the ratio of the dye content to the pigment content in the treating composition ranges from about 0.001:1 to about 1000:1, and preferably ranges from about 0.01:1 to about 10:1, and more preferably range from 0.05:1 to about 1:1.

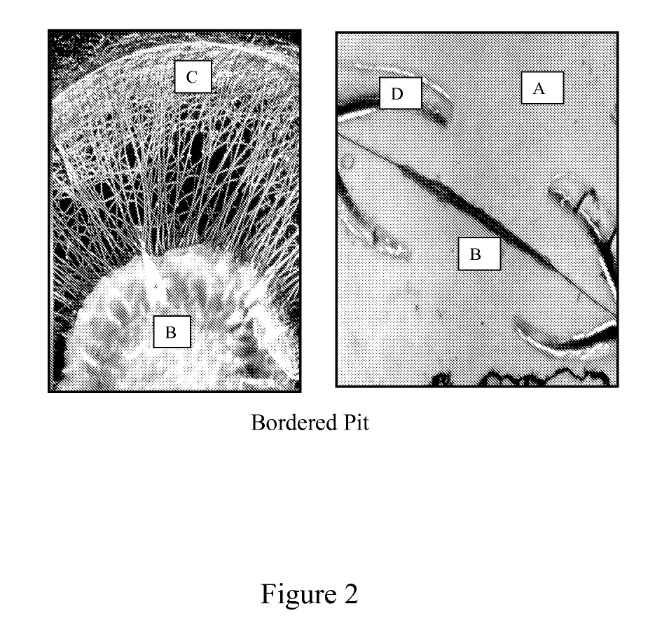

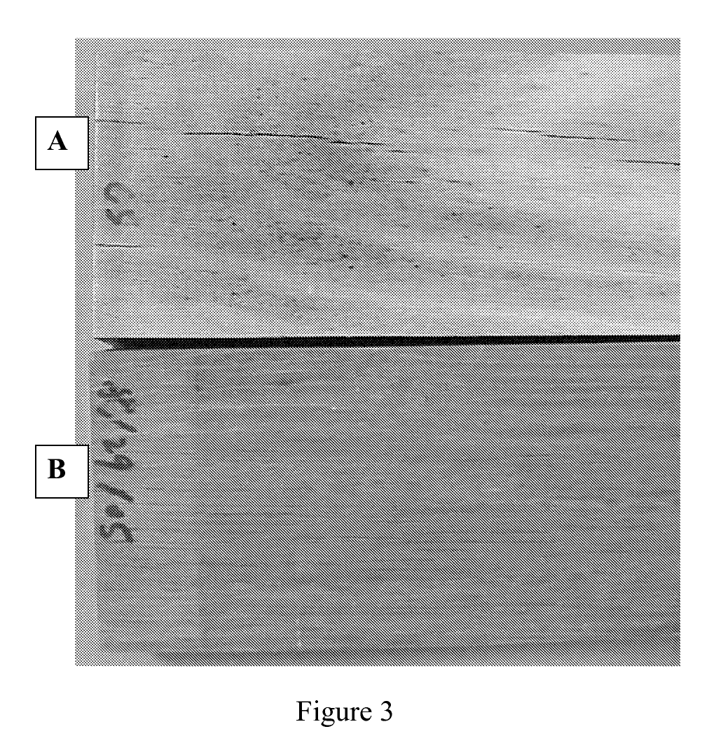

A wide range of useful colors can be imparted to wood using the process of the present invention. The color of wood treated with the preservative solutions described herein can be a variety of colors, such as grey, blue, green, brown, yellow, orange, black, red or other shades, depending upon the particular combination of the pigments, and their concentration. Dramatic improvement on the weathering properties can be achieved by incorporating the pigments into preservative systems as shown in FIGS. 3 and 4. The samples in FIG. 3 were treated with a tebuconazole based wood preservative and the samples in FIG. 4 with a quaternary ammonium compound-based wood preservative (dimethyl didecyl ammonium carbonate/bicarbonate). Specifically, the samples in FIGS. 3A and 4A were treated with the preservatives alone, while the sample in FIG. 3B was treated with the preservative plus a red-brown iron oxide-based pigment formulation and 4B with the preservative plus a iron oxide/carbon black-based green pigment formulation. The samples were then subjected to outdoor weathering. The samples treated with preservative alone showed poor weathering characteristics: delignification, surface graying, darkening, and mold growth, while the samples treated with preservatives plus pigments demonstrated excellent photo-resistance and overall color integrity.

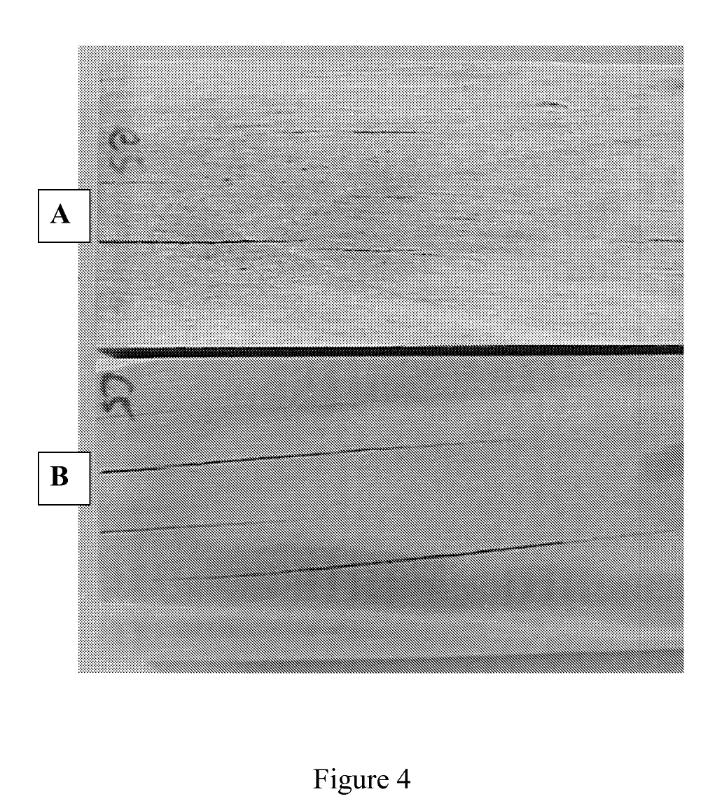

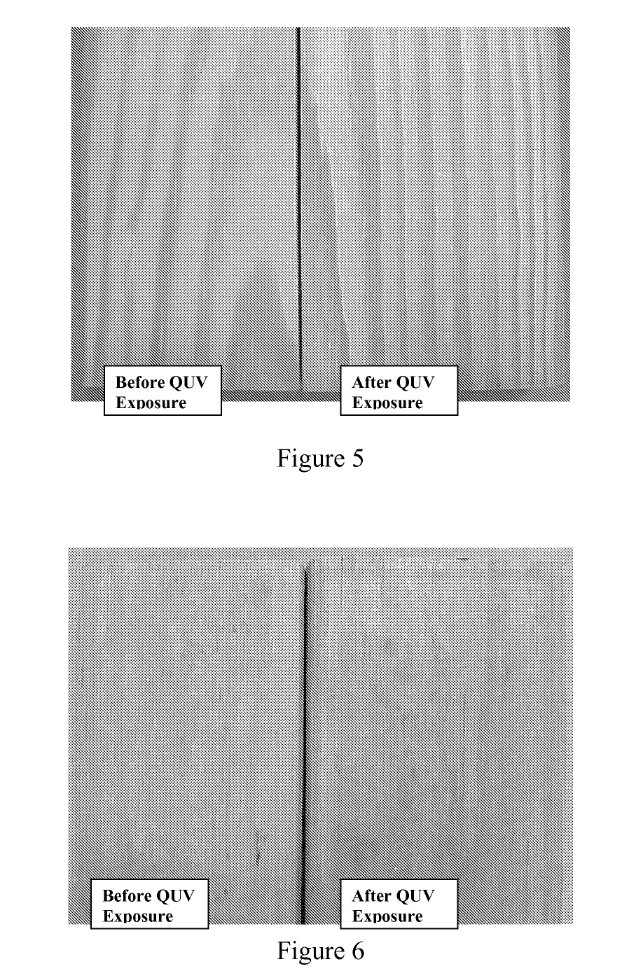

Laboratory accelerated weathering test (QUV Test: samples exposed to UV light and water spraying) also confirms that the wood samples treated with preservative plus pigment formulation demonstrated great UV photo-resistance. FIGS. 5 and 6 demonstrated the effect of QUV test on the wood samples treated with a preservative alone (tebuconazole and bifenthrin) and the preservative plus a light-brown pigment formulation (iron oxide-based), respectively. Delignification and graying were observed on the preservative alone treated sample after one month QUV weathering, while only slight color change was observed the sample treated with the preservative plus the pigment after one month QUV weathering.

The treating composition may be applied to wood by dipping, soaking, spraying, brushing, or any other means well known in the art. In a preferred embodiment, vacuum and/or pressure techniques are used to impregnate the wood in accord with this invention including the standard processes, such as the “Empty Cell” process, the “Modified Full Cell” process and the “Full Cell” process, and any other vacuum and/or pressure processes which are well known to those skilled in the art.

The standard processes are defined as described in AWPA Standard C1-03 “All Timber Products—Preservative Treatment by Pressure Processes”. In the “Empty Cell” process, prior to the introduction of preservative, materials are subjected to atmospheric air pressure (Lowry) or to higher air pressures (Rueping) of the necessary intensity and duration. In the “Modified Full Cell”, prior to introduction of preservative, materials are subjected to a vacuum of less than 77 kPa (22 inch Hg) (sea level equivalent). A final vacuum of not less than 77 kPa (22 inch Hg) (sea level equivalent) should be used. In the “Full Cell Process”, prior to introduction of preservative or during any period of condition prior to treatment, materials are subjected to a vacuum of not less than 77 kPa (22 inch Hg). A final vacuum of not less than 77 kPa (22 inch Hg) is used.

The present invention also provides a method for applying pigments to wood. In one embodiment, the method comprises the steps of treating wood with a treating fluid comprising a dispersion of micronized pigment particles according to conventional wood treatment cycles employing for example, the Full Cell or Empty Cell process, some combination thereof, or by dip or spray treatment.

It is preferable to color and preserve the wood simultaneously, however it can be desirable to treat and color the wood in two stages. Without departing from the teachings of this invention the wood may first be treated with a composition containing wood preservatives, and then contacted with a composition containing the pigment dispersion. It is also possible to apply the coloring agent to the wood initially, followed by the application of the preservative composition. The application of each component can be carried out as with the application of a two component solution.

The two step application is particularly useful in wood treatment processes in which the runoff from treatment with one component is to be collected and reused.

A variety of cellulosic products such as wood, paper, textiles, cotton and the like can be colored and preserved in accordance with this invention including hard and/or soft woods. In general, wood may thus be simultaneously colored and preserved.

Wood colored and preserved according to the method of this invention resists weathering and has many uses in the construction industry. Patio and pool decks, wood siding and beams, fence posts, garden ties and poles for outdoor or indoor use are just a few of the possible products which may incorporate wood treated according to the method described herein.

The following examples will serve to further illustrate the invention. Examples 1 through 10 demonstrate the preparation of pigment dispersion. Examples 15 through 22 demonstrate the preparation of the wood preservative treating compositions with and without the presence of pigment dispersions.

### EXAMPLE 1

1500 grams of red iron oxide, 500 g yellow iron oxide and 56 g carbon black were added to a container containing 1594.0 g of water and 350.0 g of a commercially available solution of polycarboxylate ether dispersant. The mixture was mechanically stirred for about 20 minutes and then added to a grinding mill. The sample was ground for about 2.0 hours and a stable dispersion was obtained. The particle size of the dispersed product was analyzed by Horiba LA-910 Particle Size Distribution Analyzer (PSDA). The average particle size was 0.21 microns with a distribution range of 0.04 um to 1.0 um.

### EXAMPLE 2

800 grams of red iron oxide, 200 g yellow iron oxide and 25 g carbon black were added to a container containing 795.0 g of water and 180 g of a modified poly carboxylic acid polymers dispersant. The mixture was mechanically stirred for about 20 minutes and then added to a grinding mill. The sample was ground for about 1.1 hours and a stable dispersion was obtained. The particle size of the dispersed product was analyzed by Horiba LA-910 Particle Size Distribution Analyzer (PSDA). The average particle size was 0.18 microns with a distribution range of 0.04 um to 1.5 um.

### EXAMPLE 3

750 grams of red iron oxide, 250 g yellow iron oxide and 50 g black iron oxide were added to a container containing 1270 g of water and 180 g of a modified polycarboxylate ether type of dispersant. The mixture was mechanically stirred for about 20 minutes and then added to a grinding mill. The sample was ground for about 1 hour and a stable dispersion is obtained. The particle size of the dispersed product was analyzed by Horiba LA-910 Particle Size Distribution Analyzer (PSDA). The average particle size was 0.25 microns with a distribution range of 0.005 um to 1.5 um.

### EXAMPLE 4

2000 g yellow iron oxide and 44 g carbon black were added to a container containing 2616.0 g of water and 340.0 g of a commercially available solution of polycarboxylate ether dispersant. The mixture was mechanically stirred for about 20 minutes and then added to a grinding mill. The sample was ground for about 2.5 hours and a stable dispersion obtained. The particle size of the dispersed product was analyzed by Horiba LA-910 Particle Size Distribution Analyzer (PSDA). The average particle size was preferably 0.19 microns with a distribution range of 0.01 um to 1.1 um.

### EXAMPLE 5

2000 g yellow iron oxide and 198 g red iron oxide were added to a container containing 2923.0 g of water and 374.0 g of a commercially available solution of polycarboxylate ether dispersant. The mixture was mechanically stirred for about 20 minutes and then added to a grinding mill. The sample was ground for about 2.0 hours and a stable dispersion obtained. The particle size of the dispersed product was analyzed by Horiba LA-910 Particle Size Distribution Analyzer (PSDA). The average particle size was preferably 0.18 microns with a distribution range of 0.01 um to 1.2 um.

### EXAMPLE 6

1000 g yellow iron oxide and 124 g red iron oxide were added to a container containing 1484.0 g of water and 202.0 g of a commercially available polycarboxylate ether type of dispersant. The mixture was mechanically stirred for about 20 minutes and then added to a grinding mill. The sample was ground for about 1.2 hours and a stable dispersion obtained. The particle size of the dispersed product was analyzed by Horiba LA-910 Particle Size Distribution Analyzer (PSDA). The average particle size was preferably 0.18 microns with a distribution range of 0.04 um to 1.0 um.

### EXAMPLE 7

Eight hundred and ninety grams of yellow iron oxide, 110 g red iron oxide were added to a container containing 3000 g of water and 200 g of a commercially available modified polycarboxylate ether type of dispersant. The mixture was mechanically stirred for about 20 minutes and then added to a grinding mill. The sample was ground for about 1 hour and a stable dispersion obtained. The particle size of the dispersed product was analyzed by Horiba LA-910 Particle Size Distribution Analyzer (PSDA). The average particle size was preferably 0.24 microns with a distribution range of 0.010 um to 2.0 um.

### EXAMPLE 8

Five hundred grams of organic pigment yellow PY65, 600 g of organic pigments red PR23 and 15 g organic pigment blue PB 15 were added to a container containing 3000 g of water and 450 g of a modified polyether with pigment affinic groups dispersant. The mixture is mechanically stirred for about 20 minutes and then added to a grinding mill. The sample was ground for about 1 hour and a stable dispersion obtained. The particle size of the dispersed product was analyzed by Horiba LA-910 Particle Size Distribution Analyzer (PSDA). The average particle size was 0.18 microns with a distribution range of 0.0050 um to 2.0 um.

### EXAMPLE 9

Eight hundred grams of organic pigment yellow PY 13 and 100 g of organic pigments red PR254 were added to a container containing 4000 g of water and 500 g of a modified polymer with pigment affinity group dispersant. The mixture was mechanically stirred for about 20 minutes and then added to a grinding mill. The sample was ground for about 1 hour and a stable dispersion was obtained. The particle size of the dispersed product was analyzed by Horiba LA-910 Particle Size Distribution Analyzer (PSDA). The average particle size was 0.21 microns with a distribution range of 0.001 um to 2.0 um.

### EXAMPLE 10

Five hundred grams of titanium dioxide is mixed with 450 grams of water and 50 grams of a modified polyacrylate polymer dispersants. The mixture is mechanically stirred for 5 minutes. The mixture is then placed in a grinding mill and ground for about 30 minutes. A stable dispersion is obtained with an average particle size of 0.29 microns.

### EXAMPLE 11

Southern Yellow Pine, (measuring 2″×6″×4′) was simultaneously colored and preserved by the Full Cell treatment using a 1.1% MicroPro200 solution containing 0.73% micronized copper carbonate as copper oxide and 0.37% quaternary ammonium compound (dimethyl didecyl ammonium carbonate/bicarbonate), and 0.45% pigment solids from Example 1. The wood was initially placed under a vacuum of 27″ Hg for 30 minutes followed by the addition of the treating solution. The system was then pressurized for 30 minutes at a pressure of 110 lbs. per square inch. The resulting wood, when dried, was colored reddish brown and protected against wood destroying organisms.

### EXAMPLE 12

Southern Yellow Pine, (measuring 2″×6″×4′) was simultaneously colored and preserved by a modified full cell treatment using a 1.2% MicroPro200 solution containing 0.80% micronized copper carbonate expressed as copper oxide and 0.40% quaternary ammonium compound (dimethyl didecyl ammonium carbonate/bicarbonate), and 0.50% pigment solids from Example 1. The wood was initially placed under a vacuum of 22″ Hg for 7 minutes followed by the addition of the treating solution. The system was then pressurized for 10 minutes at a pressure of 110 lbs. per square inch, followed with a 20 minutes final vacuum. The resulting wood, when dried, was colored reddish brown and protected against wood destroying organisms.

### EXAMPLE 13

Southern Yellow Pine, (measuring 2″×6″×4′) was simultaneously colored and preserved by a modified full cell treating process using a 1.0% MicroPro200 solution containing 0.667% micronized copper carbonate expressed as copper oxide and 0.333% quaternary ammonium compound (dimethyl didecyl ammonium carbonate/bicarbonate), and 0.45% pigment solids from Example 5. The wood was initially placed under a vacuum of 22″ Hg for 5 minutes followed by the addition of the treating solution. The system was then pressurized for 5 minutes at a pressure of 110 lbs. per square inch, followed by a 25 minutes final vacuum. The resulting wood, when dried, was colored cedar-brown and protected against wood destroying organisms.

### EXAMPLE 14

Southern Yellow Pine, (measuring 2″×6″×4′) was simultaneously colored and preserved by a modified full cell treating process using a 1.0% MicroPro200 solution containing 0.667% micronized copper carbonate expressed as copper oxide and 0.333% quaternary ammonium compound (dimethyl didecyl ammonium carbonate/bicarbonate), and 0.48% pigment solids from Example 5 and 0.024% of 2-(((4-Methoxyphenyl)methylhydrazono)methyl)-1,3,3-trimethyl-3H-indolium methyl sulphate dye. The wood was initially placed under a vacuum of 22″ Hg for 5 minutes followed by the addition of the treating solution. The system was then pressurized for 5 minutes at a pressure of 110 lbs. per square inch, followed by a 25 minutes final vacuum. The resulting wood, when dried, was colored brown and protected against wood destroying organisms.

### EXAMPLE 15

Southern Yellow Pine blocks (1½″×2″×6″) were simultaneously colored and preserved utilizing the Lowry Empty Cell process using a 0.5% quaternary ammonium compounds based preservative (dimethyl didecyl ammonium chloride) plus 0.49% pigment solids from Example 2 and 0.030% hydroxyethyl cellulose binder. The resulting wood was air dried to a 20% moisture content and was colored a reddish brown color uniformly distributed on the surface of the treated wood. The wood was exposed under an accelerated tester (QUV) and found to exhibit great resistance to UV photo-degradation. Laboratory accelerated agar test indicated that the treated wood resist both attacks from brown rots and white rots.

### EXAMPLE 16

50 grams of pigment concentrate from Example 5 were mixed with 3950 g dimethyldidecylammonium carbonate (DDA Quat) water solution containing 0.60% DDA Quat and 0.05% polyvinyl alcohol resin. The solution was used to treat red pine and ponderosa pine samples using the Full Cell process. The resulting wood was oven dried at 120° F. and was colored a cedar-brown color. Outdoor exposure studies indicated that the treated samples were resistant to biological deterioration and UV degradation.

### EXAMPLE 17

Southern Yellow Pine blocks (½″×2″×6″) were simultaneously colored and preserved using the Full Cell treatment with a treating composition containing 0.10% copper 8-hydroxyquinolate plus 0.50% pigment dispersion from Example 1 with 0.005% a PVA type of binder. The Southern Yellow Pine blocks were placed in a cylinder and a vacuum of 30″ Hg applied for 15 minutes, the treating composition was then added to the cylinder and the system pressurized to 100 lbs. per square inch for 30 minutes. The resulting wood, when dried, was colored a reddish brown and was protected against wood destroying organisms.

### EXAMPLE 18

Douglas fir and Hem fir wood samples were colored a brown color with a two-step process. Step I involved the treatment of wood with 0.8% dimethyldidecylammonium carbonate solution using the Full Cell process, followed by Step II treatment with a 1.0% pigment solution from Example 5. The treated wood showed bio-efficacy and color stability when exposed outside.

### EXAMPLE 19

Southern pine, red pine and ponderosa pine samples were colored a darker reddish brown color with a two-step process. Step I involved the treatment of wood with a 2.0% pigment solution from Example 2 plus 0.015% a commercially available binder using a modified Full Cell process, followed by Step II treatment with a composition containing 0.05% tebuconazole and 0.005% bifenthrin. The treated samples demonstrated uniform surface coloration. The samples also demonstrated bio-efficacy in a field test evaluation.

### EXAMPLE 20

Douglas fir and Hem fir samples were colored a darker reddish brown color with a two-step process. Step I involved the treatment of wood with a composition containing 1.0% dimethyldidecylammonium carbonate solution using the Full Cell process, followed by Step II treatment with a 1.0% pigment solution from Example 2 plus 0.01% a commercially available binder. The treated samples demonstrated uniform coloration on the surface and bio-efficacy in a field test evaluation.

### EXAMPLE 21

Southern Yellow Pine, (measuring 2″×6″×4′) was simultaneously colored and preserved by a modified full cell treating process using a preservative solution containing 0.15% tebuconazole and 0.03% bifenthrin and 0.50% pigment solids from Example 1. The wood was initially placed under a vacuum of 22″ Hg for 5 minutes followed by the addition of the treating solution. The system was then pressurized for 5 minutes at a pressure of 110 lbs. per square inch, followed by a 25 minutes final vacuum. The resulting wood, when dried, was colored reddish brown color and protected against wood destroying organisms.

### EXAMPLE 22

Southern Yellow Pine, (measuring 2″×6″×4′) was simultaneously colored and preserved by a modified full cell treating process using a preservative solution containing 0.10% tebuconazole and 0.02% bifenthrin and 0.50% pigment solids from Example 5 The wood was initially placed under a vacuum of 22″ Hg for 6 minutes followed by the addition of the treating solution. The system was then pressurized for 7 minutes at a pressure of 110 lbs. per square inch, followed by a 25 minutes final vacuum. The resulting wood, when dried, was colored cedar-brown and protected against wood destroying organisms.

The foregoing examples are intended to be merely illustrative and should not be construed or interpreted as being restrictive or otherwise limiting of the present invention.

We claim:

1. A composition for preserving wood against wood destroying organisms and coloring the wood comprising:
a) a micronized pigment component; b) a dispersant; and c) a biocide;
wherein a), and optionally, c) comprise a particulate dispersion comprising micronized particles, and wherein greater than 60 weight percent of the particles present in said composition are micronized.

2. The composition of claim 1, wherein the pigment is a micronized inorganic pigment.

3. The composition of claim 2, wherein greater than 60 weight percent of the particles of micronized inorganic pigment have sizes between 0.001 microns and 25 microns.

4. The composition of claim 2, wherein greater than 60 weight percent of the particles of micronized inorganic pigment have sizes between 0.005 to 10 microns.

5. The composition of claim 2, wherein greater than 60 weight percent of the particles of micronized inorganic pigment have sizes between 0.05 to 5 microns.

6. The composition of claim 2, wherein greater than 60 weight percent of the particles of micronized inorganic pigment have sizes between 0.05 to 1 micron.

7. The composition of claim 2, wherein the inorganic pigment comprises a metal selected from the group consisting of iron, copper, aluminum, calcium, zinc, titanium, cobalt, tin, nickel, silver, silicon, chromium, barium, bismuth, carbon black, and graphite.

8. The composition of claim 7, wherein the inorganic pigment is selected from the group consisting of iron oxide, red iron oxide, yellow iron oxide, black iron oxide, brown iron oxides, carbon black, iron hydroxide, graphite, black micaceous iron oxide, aluminum flake pigment, pearlescent pigment, calcium carbonate, calcium phosphate, calcium oxide, calcium hydroxide, bismuth oxide, bismuth hydroxide, bismuth carbonate, copper carbonate, copper hydroxide, basic copper carbonate, cupric oxide, cuprous oxide, silicon oxide, zinc carbonate, barium carbonate, barium hydroxide, strontium carbonate, zinc oxide, zinc phosphate, zinc chromate, barium chromate, chrome oxide, titanium dioxide, zinc sulfide, antimony oxide, lead chrome, and a cadmium pigment.

9. The composition of claim 1, wherein the micronized pigment is an organic pigment.

10. The composition of claim 9, wherein the micronized organic pigment selected from the group consisting of monoazo (arylide) pigments, disazo (diarylide) pigments, disazo condensation pigments, benzimidazolone pigments, beta Naphthol pigments, Naphthol pigments, metal-organic complexes, Isoindoline, Isoindolinone, quinacridone; perylene; perinone; anthraquinone; diketo-pyrrolo pyrrole; dioxazine; triacrylcarbonium; cobalt phthalocyanine, copper phthalocyanine, copper semichloro- or monochlorophthalocyanine, copper phthalocyanine, metal-free phthalocyanine, copper polychlorophthalocyanine, phthalocyanine blue, other phthalocyanine pigments; organic azo compound pigments; organic nitro compound pigments; polycyclic compound pigments, phthalocyanine pigments, quinacridone pigments, perylene pigments, perinone pigments; diketopyrrolo-pyrrole(DPP) pigments; thioindigo pigments; dioxazine pigments; quinophthalone pigments; triacrylcarbonium pigments, and diaryl pyrrolopyrole pigments.

11. The composition of claim 1, wherein the dispersant is a polymeric dispersant.

12. The composition of claim 11, wherein the polymeric dispersant is selected from group consisting of acrylic copolymers, aqueous solutions of copolymers having pigment affinity groups, polycarboxylate ethers, modified polyacrylates or modified polyacrylates having high pigment affinity, acrylic polymer emulsions, modified acrylic polymers, poly carboxylic acid polymers and their salts, modified poly carboxylic acid polymers and their salts, fatty acid modified polyester, aliphatic polyether or modified aliphatic polyether, polycarboxylate ether solutions, phosphate esters, phosphate ester-modified polymers, polyglycol ethers, modified polyglycol ethers, polyetherphosphate, modified maleic anhydride/styrene copolymers, sodium polyacrylate, sodium polymethacrylate, lignin, modified lignin and the like; modified polyether or polyester with pigment affinic groups; fatty acid derivatives; urethane copolymer or modified urethane copolymer, polyetherphosphate, modified maleic anhydride/styrene copolymer, modified polycarboxylic acids or their derivatives, acrylic acid/maleic acid copolymers, polyvinyl pyrrolidones, modified polyvinyl pyrrolidones, sulfonates, sulfonate derivatives, polymeric alkoxylates or its polymeric alkoxylate derivatives, or modified lignin.

13. The composition of claim 2, wherein the dispersant comprises 0.1 to 180% of the weight of the pigment compounds.

14. A composition as in claim 13 wherein the dispersant is preferably in the range of from about 1 to 80% of the weight of the pigment compounds.

15. A composition as in claim 14 wherein the dispersant is preferably in the range of from about 5 to 60% of the weight of the pigment compounds.

16. A composition as in claim 15 wherein the dispersant is preferably in the range of from about 10 to 30% of the weight of the pigment compounds.

17. The composition of claim 9, wherein the dispersant comprises 1 to 200% of the weight of the pigment compounds.

18. The composition as in claim 17, wherein the dispersant is preferably in the range of from about 10 to 80% of the weight of the pigment compounds.

19. The composition as in claim 18 wherein the dispersant is preferably in the range of from about 30 to 70% of the weight of the pigment compounds.

20. The composition of claim 1, wherein the said biocide is an inorganic biocide.

21. The composition of claim 20, wherein the inorganic biocide comprises a compound comprising a metal selected from the group consisting of a compound of copper, boron, zinc, cobalt, cadmium, silver, nickel, and tin.

22. The composition of claim 21, wherein the inorganic biocide is a copper compound, a zinc compound or a boron compound.

23. The composition of claim 22, wherein the biocide is copper hydroxide, copper oxide, copper carbonate, basic copper carbonate, copper oxychloride, copper 8-hydroxyquinolate, copper dimethyldithiocarbamate, copper omadine or copper borate.

24. The composition of claim 22, wherein the biocide is zinc oxide, zinc phosphate, zinc borate or zinc carbonate.

25. The composition of claim 20, where the inorganic biocide is micronized.

26. The composition of claim 1, wherein the biocide is an organic biocide.

27. The composition of claim 26, wherein the biocide is a triazole or an imidazole.

28. The composition of claim 27, wherein the biocide is tebuconazole; cyproconazole; propiconazole; hexaconazole; 1-[[2-(2,4-dichlorophenyl)-1,3-dioxolan-2-yl]methyl]-1H-1,2,4-triazole; cis-trans-3-chloro-4-[4-methyl-2-(1H-1,2,4-triazol-1-ylmethyl)-1,3-dioxolan-2-yl]phenyl 4-chlorophenyl ether; (RS)-2-(4-fluorophenyl)-1-(1H-1,2,4-triazol-1-yl)-3-(trimethylsilyl)propan-2-ol; or 2-(2,4-difluorophenyl)-1-(1H-1,2,4-triazole-1-yl)-3-trimethylsilyl-2-propanol.

29. The composition of claim 26, wherein the organic biocide is a quaternary ammonium compound.

30. The composition of claim 29, wherein the quaternary ammonium compound is selected from the group consisting of didecyldimethylammonium chloride, didecyldimethylammonium carbonate/bicarbonate, alkyldimethylbenzylammonium chloride, alkyldimethylbenzylammonium carbonate/bicarbonate, didodecyldimethylammonium chloride, didodecyldimethylammonium carbonate/bicarbonate, didodecyldimethylammonium propionate, and N,N-didecyl-N-methyl-poly(oxyethyl)ammonium propionate.

31. The composition of claim 26, wherein the organic biocide is an isothiazolone compound.

32. The composition of claim 31, where in the isothiazolone compound is methylisothiazolinone, chloromethylisothiazolinone, 4,5-Dichloro-2-n-octyl-3(2H)-isothiazolone, 1,2-benzisothiazolin-3-one, or 2-octyl-3-isothiazolone.

33. The composition of claim 26, wherein the organic biocide is synthetic pyrethroid compound.

34. The composition of claim 33, where the synthetic pyrethroid compound is acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin, transfluthrin, etofenprox, flufenprox, halfenprox, protrifenbute, or silafluofen.

35. The composition of claim 26, where the organic biocide is iodopropynyl butylcarbamate, chlorothalonil, 2-(thiocyanatomethylthio) benzothiazole; alkoxylated diamines and carbendazim, fipronil or imidachloprid.

36. The composition of claim 26, where the organic biocide is micronized.

37. The composition of claim 1 further comprising a resin binder.

38. The composition of claim 1 further comprising a dye.

39. The composition of the claim 38, wherein the dye is a acid dye, basic dye, direct dye, or reactive dye.

40. A method for coloring wood or a wood product comprising the step of impregnating the product with the wood coloring composition comprising:
a) a pigment component; and
b) a dispersant;
wherein a) comprises a dispersion of particles comprising micronized particles, and wherein greater than 60 weight percent of the particles present in said dispersion are micronized.

41. The method of claim 40, wherein the pigment is a micronized inorganic pigment.

42. The method of claim 41, wherein greater than 60 weight percent of the particles of micronized inorganic pigment have sizes between 0.001 microns and 25 microns.

43. The method of claim 41, wherein greater than 60 weight percent of the particles of micronized inorganic pigment have sizes between 0.005 to 10 microns.

44. The method of claim 41, wherein greater than 60 weight percent of the particles of micronized inorganic pigment have sizes between 0.05 to 5 microns.

45. The method of claim 41, wherein greater than 60 weight percent of the particles of micronized inorganic pigment have sizes between 0.05 to 1 micron.

46. The method of claim 41, wherein the inorganic pigment comprises a metal selected from the group consisting of iron, copper, aluminum, calcium, zinc, titanium, cobalt, tin, nickel, silver, silicon, chromium, barium, bismuth, carbon black, and graphite.

47. The method of claim 46, wherein the inorganic pigment is selected from the group consisting of iron oxide, red iron oxide, yellow iron oxide, black iron oxide, brown iron oxides, carbon black, iron hydroxide, graphite, black micaceous iron oxide, aluminum flake pigment, pearlescent pigment, calcium carbonate, calcium phosphate, calcium oxide, calcium hydroxide, bismuth oxide, bismuth hydroxide, bismuth carbonate, copper carbonate, copper hydroxide, basic copper carbonate, cupric oxide, cuprous oxide, silicon oxide, zinc carbonate, barium carbonate, barium hydroxide, strontium carbonate, zinc oxide, zinc phosphate, zinc chromate, barium chromate, chrome oxide, titanium dioxide, zinc sulfide, antimony oxide, lead chrome, and a cadmium pigment.

48. The method of claim 40, wherein the micronized pigment is an organic pigment.

49. The method of claim 48, wherein the micronized organic pigment selected from the group consisting of monoazo (arylide) pigments, disazo (diarylide) pigments, disazo condensation pigments, benzimidazolone pigments, beta Naphthol pigments, Naphthol pigments, metal-organic complexes, Isoindoline, Isoindolinone, quinacridone; perylene; perinone; anthraquinone; diketo-pyrrolo pyrrole; dioxazine; triacrylcarbonium; cobalt phthalocyanine, copper phthalocyanine, copper semichloro- or monochlorophthalocyanine, copper phthalocyanine, metal-free phthalocyanine, copper polychlorophthalocyanine, phthalocyanine blue, other phthalocyanine pigments; organic azo compound pigments; organic nitro compound pigments; polycyclic compound pigments, phthalocyanine pigments, quinacridone pigments, perylene pigments, perinone pigments; diketopyrrolo-pyrrole(DPP) pigments; thioindigo pigments; dioxazine pigments; quinophthalone pigments; triacrylcarbonium pigments, and diaryl pyrrolopyrole pigments.

50. The method of claim 40, wherein the dispersant is a polymeric dispersant.

51. The method of claim 50, wherein the polymeric dispersant is selected from group consisting of acrylic copolymers, aqueous solutions of copolymers having pigment affinity groups, polycarboxylate ethers, modified polyacrylates or modified polyacrylates having high pigment affinity, acrylic polymer emulsions, modified acrylic polymers, poly carboxylic acid polymers and their salts, modified poly carboxylic acid polymers and their salts, fatty acid modified polyester, aliphatic polyether or modified aliphatic polyether, polycarboxylate ether solutions, phosphate esters, phosphate ester-modified polymers, polyglycol ethers, modified polyglycol ethers, polyetherphosphate, modified maleic anhydride/styrene copolymers, sodium polyacrylate, sodium polymethacrylate, lignin, modified lignin and the like; modified polyether or polyester with pigment affinic groups; fatty acid derivatives; urethane copolymer or modified urethane copolymer, polyetherphosphate, modified maleic anhydride/styrene copolymer, modified polycarboxylic acids or their derivatives, acrylic acid/maleic acid copolymers, polyvinyl pyrrolidones, modified polyvinyl pyrrolidones, sulfonates, sulfonate derivatives, polymeric alkoxylates or its polymeric alkoxylate derivatives, or modified lignin.

52. The method of claim 41, wherein the dispersant comprises 0.1 to 180% of the weight of the pigment compounds.

53. A method as in claim 52 wherein the dispersant is preferably in the range of from about 1 to 80% of the weight of the pigment compounds.

54. A method as in claim 53 wherein the dispersant is preferably in the range of from about 5 to 60% of the weight of the pigment compounds.

55. A method as in claim 54 wherein the dispersant is preferably in the range of from about 10 to 30% of the weight of the pigment compounds.

56. The method of claim 48, wherein the dispersant comprises 1 to 200% of the weight of the pigment compounds.

57. The method as in claim 56, wherein the dispersant is preferably in the range of from about 10 to 80% of the weight of the pigment compounds.

58. The method as in claim 57 wherein the dispersant is preferably in the range of from about 30 to 70% of the weight of the pigment compounds.

59. The method of claim 40, wherein the wood or wood product is additionally impregnated with a biocide, wherein 1) the composition additionally comprises the biocide, or 2) the method additionally comprises a step which comprises impregnation with a biocide; and wherein the biocide impregnation step takes place prior to or after the impregnation of the wood or wood product with the pigment.

60. The method of claim 59, wherein the biocide is an inorganic biocide.

61. The method of claim 60, wherein the inorganic biocide comprises a compound comprising a metal selected from the group consisting of a compound of copper, boron, zinc, cobalt, cadmium, silver, nickel, and tin.

62. The method of claim 61, wherein the inorganic biocide is a copper compound, a zinc compound or a boron compound.

63. The method of claim 62, wherein the biocide is copper hydroxide, copper oxide, copper carbonate, basic copper carbonate, copper oxychloride, copper 8-hydroxyquinolate, copper dimethyldithiocarbamate, copper omadine or copper borate.

64. The method of claim 62, wherein the biocide is zinc oxide, zinc phosphate, zinc borate, or zinc carbonate.

65. The method of claim 60, where the inorganic biocide is micronized.

66. The method of claim 59, wherein the biocide is an organic biocide.

67. The method of claim 66, wherein the biocide is a triazole or an imidazole.

68. The composition of claim 67, wherein the biocide is tebuconazole; cyproconazole; propiconazole; hexaconazole; 1-[[2-(2,4-dichlorophenyl)-1,3-dioxolan-2-yl]methyl]-1H-1,2,4-triazole; cis-trans-3-chloro-4-[4-methyl-2-(1H-1,2,4-triazol-1-ylmethyl)-1,3-dioxolan-2-yl]phenyl 4-chlorophenyl ether; (RS)-2-(4-fluorophenyl)-1-(1H-1,2,4-triazol-1-yl)-3-(trimethylsilyl)propan-2-ol; or 2-(2,4-difluorophenyl)-1-(1H-1,2,4-triazole-1-yl)-3-trimethylsilyl-2-propanol.

69. The method of claim 66, wherein the organic biocide is a quaternary ammonium compound.

70. The method of claim 69, wherein the quaternary ammonium compound is selected from the group consisting of didecyldimethylammonium chloride, didecyldimethylammonium carbonate/bicarbonate, alkyldimethylbenzylammonium chloride, alkyldimethylbenzylammonium carbonate/bicarbonate, didodecyldimethylammonium chloride, didodecyldimethylammonium carbonate/bicarbonate, didodecyldimethylammonium propionate, and N,N-didecyl-N-methyl-poly(oxyethyl)ammonium propionate.

71. The method of claim 66, wherein the organic biocide is an isothiazolone compound.

72. The method of claim 71, where in the isothiazolone compound is methylisothiazolinone, chloromethylisothiazolinone, 4,5-Dichloro-2-n-octyl-3(2H)-isothiazolone, 1,2-benzisothiazolin-3-one, or 2-octyl-3-isothiazolone.

73. The method of claim 66, wherein the organic biocide is synthetic pyrethroid compound.

74. The method of claim 73, where the synthetic pyrethroid compound is acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin, transfluthrin, etofenprox, flufenprox, halfenprox, protrifenbute, or silafluofen.

75. The method of claim 66, where the organic biocide is iodopropynyl butylcarbamate, chlorothalonil, 2-(thiocyanatomethylthio) benzothiazole; alkoxylated diamines and carbendazim, fipronil or imidachloprid.

76. The method of claim 66, where the organic biocide is micronized.

77. The method of claim 40 further comprising a resin binder.

78. The method of claim 40 further comprising a dye.

79. The method of claim 78, wherein the dye is an acid dye, basic dye, direct dye, or reactive dye.

80. Wood or a wood product having distributed through at least a portion thereof a composition comprising:
a) a dispersion of pigment particles; and
b) a dispersant,
wherein greater than 60 weight percent said particles are micronized.

81. Wood or a wood product as in claim 80 wherein the wood is a coniferous wood.

82. Wood or a wood product as in claim 80 wherein said composition additionally comprises a particulate biocide.

83. Wood or a wood product as in claim 80 wherein said particulate biocide comprises copper or a copper compound.

84. Wood or a wood product as in claim 83 wherein greater than 60 weight percent of the pigment and biocide particles present in said composition are micronized.

85. Wood or a wood product as in claim 84 wherein said biocide comprises one or more inorganic and/or organic biocides selected from the group consisting of tebuconazole, bifenthrin, dimethyl didecyl ammonium carbonate/bicarbonate and dimethyl didecyl ammonium chloride, propiconazole, cyproconazole, 4,5Dichloro-2-N-Octyl-4-isothiazolin-3-one (rh-287) imidacloprid, fipronil, permethrin, and cypromethrin.

86. Wood or a wood product as in claim 84, further comprising a resin binder.

87. Wood or a wood product as in claim 84 wherein a) comprises one or more organic pigments.

88. Wood or a wood product as in claim 84 wherein said dispersant comprises one or more polymers selected from the group consisting of a modified maleic anhydride/styrene copolymer, acrylic acid/maleic acid copolymer and combinations thereof.

89. Wood or a wood product as in claim 88 wherein the dispersant is preferably in the range of from about 1 to 200% of the weight of the pigment compounds.

90. Wood or a wood product as in claim 88 wherein the dispersant is preferably in the range of from about 10 to 80% of the weight of the pigment compounds.

91. Wood or a wood product as in claim 88 wherein the dispersant is preferably in the range of from about 30 to 70% of the weight of the pigment compounds.

92. Wood or a wood product as in claim 84 wherein a) comprises one or more inorganic pigments.

93. Wood or a wood product as in claim 92 wherein said dispersant comprises one or more polymers selected from the group consisting of a polycarboxylate ether, a modified poly carboxylic acid polymers or its salts, a modified polyether or polyester with pigment affinic groups.

94. Wood or a wood product as in claim 93 wherein the dispersant is preferably in the range of from about 1 to 200% of the weight of the pigment compounds.

95. Wood or a wood product as in claim 93 wherein the dispersant is preferably in the range of from about 0.1 to 180% of the weight of the pigment compounds.

96. Wood or a wood product as in claim 93 wherein the dispersant is preferably in the range of from about 1 to 80% of the weight of the pigment compounds.

97. Wood or a wood product as in claim 93 wherein said dispersant is preferably in the range of from about 5 to 60% of the weight of the pigment compounds.

98. Wood or a wood product as in claim 93 wherein said dispersant is preferably in the range of from about 10 to 30% of the weight of the pigment compounds.

99. Wood or a wood product as in claim 80 wherein a) comprises a pigment selected from the group consisting of carbon black, graphite, iron oxide, black micaceous iron oxide, iron hydroxide, zinc oxide, titanium oxide, titanium dioxide, aluminum oxide and aluminum hydroxide.

100. Wood or a wood product as in claim 99 wherein a) comprises iron oxide selected from the group consisting of red iron oxides, yellow iron oxides, black iron oxides and brown iron oxides.

101. Wood or a wood product as in claim 80 wherein a) comprises one or more organic pigments yellow, red, orange, green, blue, black or brown.

102. Wood or a wood product as in claim 80 having a dye distributed through at least a portion thereof.
